(19) United States
(12) Patent Application Publication
(10) Pub. No.: US 2005/0158350 A1
(43) Pub. Date: Jul. 21, 2005

(54) Use of licochalcone a or of an extract of radix glycyrrhizae inflatae that contains licochalcone a against postinflammatory hyperpigmentation

(76) Inventors: Heiner Max, Hamburg (DE); Rainer Wolber, Hamburg (DE); Christopher Mummert, Bienenbuttel (DE); Ludger Kolbe, Dohren (DE); Karen Tom Dieck, Hamburg (DE); Ursula Wensorra, Hamburg (DE)

Correspondence Address: GREENBLUM & BERNSTEIN, P.L.C., 1950 ROLAND CLARKE PLACE, RESTON, VA, 20191, US

(21) Appl. No.: 10/966,036

(22) Filed: Oct. 18, 2004

(30) Foreign Application Priority Data
Nov. 10, 2003 (DE) 103 52 369.3

(51) Int. Cl. A61K 6/00; A61K 7/00; A61K 31/12; A61K 35/78

(52) U.S. Cl. 424/401; 424/757; 514/685

(57) ABSTRACT

Use of licochalcone A or an extract of radix glycyrrhizae inflatae containing licochalcone A in cosmetic or dermatological preparations for the treatment and prophylaxis of the symptoms of intrinsic and/or extrinsic aging of the skin and for the treatment and prophylaxis of the harmful effects of ultraviolet radiation on the skin.

### CROSS-REFERENCE TO RELATED APPLICATIONS

The present application claims priority under 35 U.S.C. § 119 of German Patent Application No. 103 52 369.3 filed on Nov. 10, 2003, the disclosure of which is expressly incorporated by reference herein in its entirety.

The present invention relates to cosmetic or dermatological preparations containing active substances for the care and protection of the skin, in particular sensitive skin and particularly primarily skin aged or aging through intrinsic and/or extrinsic factors and the use of such active substances and combinations of such substances in the field of cosmetic and dermatological skin care.

Cosmetic skin care is to be understood primarily as meaning that the natural function of the skin as a barrier against environmental influences (e.g., dirt, chemicals, microorganisms) and against the loss of substances intrinsic to the body (e.g., water, natural fats, electrolytes) is strengthened or restored.

Impairment of this function may lead to increased resorption of toxic or allergenic substances or to attack by microorganisms, resulting in toxic or allergic skin reactions.

Another aim of skin care is to compensate for the loss by the skin of sebum and water caused by daily washing. This is particularly important if the natural regeneration ability is inadequate. Furthermore, skin care products should protect against environmental influences, in particular against sun and wind, and delay skin aging.

The factors which are responsible for skin pigmentation are the melanocytes which are found in the lowest layer of the epidermis, the stratum basale, in addition to the basal cells as pigment-forming cells which, depending on the skin type, occur either individually or in clusters of varying size.

Melanocytes contain melanosomes as characteristic cell organelles in which melanin is formed. These form melanin at higher rates, i.a., when stimulated by UV radiation. The melanin is transported via the living layers of the epidermis (keratinocytes) eventually into the horny layer (corneocytes) and leads to a more or less pronounced brownish or brown-black skin color.

Melanin is the end product of an oxidative process in which tyrosine is converted with the aid of the enzyme tyrosinase, via several intermediates to the brown to brown-black eumelanins (DHICA and DHI melanin) or with the involvement of sulfur-containing compounds to reddish pheomelanin. DHICA and DHI melanin are formed through the common intermediates dopaquinone and dopachrom. The latter, partially with the involvement of further enzymes, is converted either into indole-5,6-quinone carboxylic acid or into indole-5,6-quinone, through which the two cited eumelanins are formed.

The formation of pheomelanin occurs, i.a., through the intermediate products dopaquinone and cysteinyl dopa. The expression of melanin-synthesizing enzymes is controlled through a specific transcription factor (microphthalmia-associated transcription factor, MITF). In addition to the described enzymatic processes of melanin synthesis, other proteins are also important in melanosomes for melanogenesis. The so-called p-protein seems to play an important part here, though the exact function is still unclear.

In addition to the process of melanin synthesis in the melanocytes described above, the transfer of melanosomes, their stay in the epidermis and their breakdown and the breakdown of melanin is of crucial importance for the pigmentation of the skin. It was possible to show that the PAR-2-receptor is important for the transport of the melanosomes from the melanocytes into the keratinocytes (M. Seiberg et al., 2000, J. Cell: Sci., 113:3093-101).

Furthermore, the size and shape of the melanosomes have an effect on their light-scattering properties and thus the appearance of the skin in terms of color. Thus large, spheroid melanosomes that are present individually are found more among black Africans, whereas smaller melanosomes that are present in groups are found among Caucasians.

Hyperpigmentation problems of the skin have a variety of causes and/or are accompanying symptoms of a large number of biological processes, for example, UV radiation (for example, freckles, ephelides), genetic predisposition, abnormal pigmentation of the skin in the course of wound healing or wound scarring (post-inflammatory hyperpigmentation) or skin ageing (for example, lentigines seniles).

After inflammatory reactions, the pigmentation system of the skin reacts with partially contrary reactions. Both postinflammatory hyper- and hypopigmentations can occur. Postinflammatory hypomelanoses often occur, i.a., in connection with atopy, lupus erythematosus and psoriasis. The different forms of reaction of the pigmentation system of human skin as a result of inflammatory symptoms are understood only to a very incomplete extent.

Problems with postinflammatory hyperpigmentation often occur with darker skin types. The problem of pseudofollikulitis barbae, which is associated with cosmetically undesirable abnormal pigmentation or causes the same, is known in particular among colored males. Forms of melasma which occur in the face and decollete area in particular among women of Asian descent, and various forms of irregular pigmentation of the skin are also included among post-inflammatory hyperpigmentations. Furthermore, dark eye rings are also considered as a form of post-inflammatory hyperpigmentations, the underlying inflammation usually occurring subclinically.

In many cases such postinflammatory abnormal pigmentation is further intensified by the effect of sunlight (UV light) without a UV-induced inflammation (sunburn) occurring.

Active ingredients and preparations which counteract skin pigmentation are known. In practice, use is made essentially of preparations based on hydroquinone although, on the one hand, these only show their effect after application for several weeks and, on the other hand, application thereof for an excessively long time is risky for toxicological reasons. Albert Kligman et al. developed a so-called triformula representing a combination of 0.1% tretinoin, 5.0% hydroquinone, 0.1% dexamethasone (A. Kligman, 1975, Arch. Dermatol. 111:40-48). However, this formula is also very controversial because of possible irreversible changes in the pigmentation system of the skin.

Furthermore, skin peeling methods (chemical and mechanical peelings) are used, but they often result in inflammatory reactions and can even lead to increased instead of reduced pigmentation due to post-inflammatory hyperpigmentations that occur afterwards. All these usual methods that are used for treating post-inflammatory hyperpigmentations are characterized by radical side effects.

It was therefore the object of the following invention to remedy the disadvantages of the prior art.

In particular in view of the hitherto not completely understood reactions of the pigmentation system of the skin, it was shown completely surprisingly that the use of licochalcone A or an extract of radix glycyrrhizae inflatae containing licochalcone A is extremely effective in cosmetic or dermatological preparations for the treatment and prophylaxis of postinflammatory skin conditions and thus contributes to a more even pigmentation of the skin.

In a particularly preferred embodiment this applies to the treatment of the following hyperpigmentation conditions: postinflammatory hyperpigmentation after inflammatory reactions, in particular those connected with shaving, melasma, uneven skin tone in particular as a result of excessive exposure to sunlight. It has been shown that in a particularly preferred embodiment licochalcone A or extracts containing the same are used in combination with UV filters. In addition to the prevention and treatment of postinflammatory hyperpigmentations using the preparations according to the invention as a particularly preferred embodiment, in a preferred embodiment the formulations according to the invention also proved to be effective in the treatment of hypopigmentations.

Postinflammatory hyperpigmentations due to pseudofollikularis barbae and melasma should be mentioned as particularly preferred areas of indications.

It was therefore the object of the invention to find ways of avoiding the disadvantages of the prior art. In particular the effect of eliminating the damage associated with the endogenous, chronological and exogenous skin aging and the prophylaxis should be lasting, sustained and without the risk of side effects.

The object of the present invention was to overcome these disadvantages.

It was surprisingly found that the use of licochalcone A or of an extract of radix glycyrrhizae inflatae containing licochalcone A in cosmetic or dermatological preparations for the treatment and prophylaxis of the symptoms of intrinsic and/or extrinsic skin aging and for the treatment and prophylaxis of the harmful effects of ultraviolet radiation on the skin remedies the disadvantages of the prior art.

A use according to the invention that is in particular advantageous is characterized in that the preparations contain 0.0001 to 5% by weight, in particular 0.001 to 1% by weight, very particularly 0.005 to 0.15% by weight of licochalcone A, based on the total weight of the preparation.

Furthermore, a use according to the invention is in particular advantageous which is characterized in that the preparations contain 0.001 to 10% by weight, in particular 0.05 to 5% by weight, very particularly 0.01 to 2% by weight of one or more ethoxylated or propoxylated raw materials, based on the total weight of the preparation.

Furthermore, a use according to the invention is in particular advantageous which is characterized in that the preparations contain 0.001 to 10% by weight, in particular 0.05 to 5% by weight, very particularly 0.01 to 2% by weight of one or more polyols, based on the total weight of the preparation.

Furthermore, a use according to the invention is in particular advantageous which is characterized in that the preparations contain licochalcone as a constituent of vegetable extracts, in particular of radix glycyrrhizae inflatae.

The plant species glycyrrhiza inflata, like the licorice glycyrrhiza glabra officinal in Europe, belongs to the genus glycyrrhiza that belongs to the fabaceae (pea plants) plant family. The drug radix glycyrrhizae inflatae, i.e., the root of the plant, is, e.g., common in eastern medicine. The use of the drug as an anti-inflammatory agent is likewise known.

One constituent of the aqueous extract of radix glycyrrhizae inflatae is licochalcone A, which is characterized by the following structural formula:

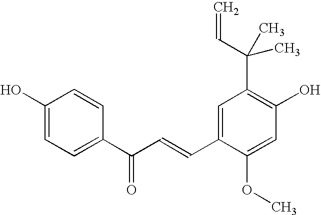

It is assumed that this substance, possible in synergy with the other constituents of the extract, plays a part in the effect according to the invention.

According to the invention, it is advantageous if the cosmetic or dermatological preparations contain 0.001 to 10% by weight, in particular 0.05 to 5% by weight, very particularly 0.01 to 2% by weight of an aqueous extract of radix glycyrrhizae inflatae based on the total weight of the preparation.

According to the invention, it is advantageous if the cosmetic or dermatological preparations contain 0.001% to 10% by weight, in particular 0.05% to 5% by weight, very particularly 0.01% to 2% by weight of one or more polyols, based on the total weight of the preparation.

It is advantageous in particular to select butylene glycol as the polyol.

It is very particularly advantageous to start from an extract that is sold by Maruzen under the name Polyol Soluble Licorice Extract P-U.

It is furthermore advantageous to use licochalcone A in other vehicle systems in a concentration of 0.0001% to 5% by weight, in particular 0.001% to 1% by weight, very particularly 0.005% to 0.05% by weight.

According to the invention, customary antioxidants can be used in preparations which contain the active substance.

The antioxidants are advantageously chosen from the group consisting of amino acids (for example, glycine, histidine, tyrosine, tryptophan) and derivatives thereof, imidazoles (for example, urocanic acid) and derivatives thereof, peptides, such as D,L-carnosine, D-carnosine, L-camosine and derivatives thereof (for example, anserine), carotenoids, carotenes (for example, α-carotene, β-carotene, lycopene) and derivatives thereof, lipoic acid and derivatives thereof (for example, dihydrolipoic acid), aurothioglucose, propylthiouracil and other thiols (for example, thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example, buthionine-sulfoximines, homocysteine-sulfoximine, buthionine sulfones, penta-, hexa- and heptathionine-sulfoximine) in very low tolerated doses (for example, pmol to μmol/kg), and furthermore (metal) chelating agents (for example, α-hydroxy-fatty acids, palmitic acid, phytic acid, lactoferrin), α-hydroxy acids (for example, citric acid, lactic acid, malic acid), humic acid, bile acid, bile extracts, bilirubin, biliverdin, EDTA, EGTA and derivatives thereof, unsaturated fatty acids and derivatives thereof (for example, γ-linolenic acid, linoleic acid, oleic acid), folic acid and derivatives thereof, alanine diacetic acid, flavonoids, polyphenols, catechols, vitamin C and derivatives (for example, ascorbyl palmitate, Mg ascorbyl phosphate, ascorbyl acetate), tocopherols and derivatives (for example, vitamin E acetate), and coniferyl benzoate of benzoin resin, rutic acid and derivatives thereof, ferulic acid and derivatives thereof, butylated hydroxytoluene, butylated hydroxyanisole, nordihydroguaiac resin acid, nordihydroguaiaretic acid, trihydroxybutyrophenone, uric acid and derivatives thereof, mannose and derivatives thereof, zinc and derivatives thereof (for example, ZnO, ZnSO₄), selenium and derivatives thereof (for example, selenium methionine), stilbenes and derivatives thereof (for example, stilbene oxide, trans-stilbene oxide) and the derivatives of these active substances mentioned which are suitable according to the invention (salts, esters, ethers, sugars, nucleotides, nucleosides, peptides and lipids).

The amount of the antioxidants (one or more compounds) in the preparations is preferably 0.001% to 30% by weight, particularly preferably 0.05-20% by weight, in particular 1-10% by weight, based on the total weight of the preparation.

The prophylaxis or the cosmetic or dermatological treatment with the active substance used according to the invention or with the cosmetic or topical dermatological preparations having an effective content of active substance used according to the invention is carried out in the usual manner, namely by applying the active substance used according to the invention or the cosmetic or topical dermatological preparations having an effective content of active ingredient used according to the invention to the affected areas of the skin.

The active substance used according to the invention can advantageously be incorporated into customary cosmetic and dermatological preparations which may be in a variety of forms. They can, for example, be a solution, an emulsion of the water-in-oil (W/O) type or of the oil-in-water (O/W) type, or a multiple emulsions, for example of the water-in-oil-in-water (W/O/W) type or oil-in-water-in-oil (O/W/O) type, a hydrodispersion or lipodispersion, a gel, a solid stick or an aerosol.

Emulsions according to the invention for the purposes of the present invention, e.g., in the form of a cream, a lotion, a cosmetic milk, are advantageous and comprise, for example, fats, oils, waxes and/or other fatty substances, and water and one or more emulsifiers as are customarily used for this type of formulation.

It is also possible and advantageous for the purposes of the present invention to incorporate the active substance used according to the invention into aqueous systems or surfactant preparations for cleansing the skin and the hair.

One of skill in the art is of course aware that sophisticated cosmetic compositions are mostly inconceivable without the customary auxiliaries and additives. Examples thereof include bodying agents, fillers, perfume, dyes, emulsifiers, additional active substances, such as vitamins or proteins, light-protection agents, stabilizers, insect repellents, alcohol, water, salts, and antimicrobially, proteolytically or keratolytically active substances etc.

Corresponding requirements apply mutatis mutandis to the formulation of medical preparations.

Medical topical compositions for the purposes of the present invention generally comprise one or more medicaments in an effective concentration. For the sake of simplicity, to make a clear distinction between cosmetic and medical application and corresponding products, reference is made to the legal provisions of the Federal Republic of Germany (e.g., Cosmetics Directive, Foods and Drugs Act).

In this connection, it is likewise advantageous to add the active substance used according to the invention as an additive to preparations which already comprise other active substances for other purposes.

Accordingly, for the purposes of the present invention, depending on their formulation, cosmetic or topical dermatological compositions can be used, for example, as skin protection cream, cleansing milk, sunscreen lotion, nourishing cream, day or night cream, etc. In some instances it may be possible and advantageous to use the compositions according to the invention as bases for pharmaceutical formulations.

Also favorable in some instances may be cosmetic and dermatological preparations which are in the form of a sunscreen. In addition to the active substance used according to the invention, these preferably additionally comprise at least one WVA filter substance and/or at least one UVB filter substance and/or at least one inorganic pigment.

It is, however, also advantageous for the purposes of the present invention to provide cosmetic and dermatological preparations whose main purpose is not protection against sunlight, but which nevertheless have a content of UV protection substances. Thus, for example, UVA and/or UVB filter substances are usually incorporated into day creams.

Preparations according to the invention can advantageously contain substances which absorb UV radiation in the UVB range, the total amount of filter substances being, for example, 0.1% by weight to 30% by weight, preferably 0.5. to 10% by weight, in particular 1 to 6% by weight, based on the total weight of the preparations.

The UVB filters can be oil-soluble or water-soluble. Examples of oil-soluble substances are:
3-benzylidene camphor and derivatives thereof, e.g., 3-(4-methylbenzylidene) camphor,
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
esters of salicylic acid, preferably 2-ethylhexyl salicylate, 4-isopropylbenzyl salicylate, homomenthyl salicylate;
derivatives of benzophenone, preferably 2-hydroxy-4-methoxybenzophenone, 2-hydroxy-4-methoxy-4′-methylbenzophenone, 2,2′-dihydroxy-4-methoxybenzo-phenone;
esters of benzalmalonic acid, preferably di(2-ethylhexyl) 4-methoxybenzalmalonate;
2,4,6-trianilino(p-carbo-2′-ethyl-1′-hexyloxy)-1,3,5-triazine.

Advantageous water-soluble substances are:
2-phenylbenzimidazole-5-sulfonic acid and salts thereof, e.g., sodium, potassium or triethanolammonium salts;
sulfonic acid derivatives of benzophenones, preferably 2-hydroxy-4-methoxybenzophenone-5-sulfonic acid and its salts;
sulfonic acid derivatives of 3-benzylidene camphor, such as, for example, 4-(2-oxo-3-bomylidenemethyl)benzenesulfonic acid, 2-methyl-5-(2-oxo-3-bomylidenemethyl)sulfonic acid and its salts.

The list of said UVB filters which can be used according to the invention is of course not intended to be limiting.

The subject matter of the invention is also the combination of a WVA filter according to the invention with a UVB filter or a cosmetic or dermatological preparation according to the invention which also comprises a UVB filter.

It can also be advantageous to use UVA filters which are customarily present in cosmetic and/or dermatological preparations in preparations according to the invention. Such filter substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. Preparations which comprise these combinations are also subject of the invention. It is possible to use the same amounts of UVA filter substances which have been given for UVB filter substances.

Cosmetic and/or dermatological preparations for the purposes of the present invention can also comprise inorganic pigments which are customarily used in cosmetics for protecting the skin against UV rays. These are oxides of titanium, zinc, iron, zirconium, silicon, manganese, aluminum, cerium and mixtures thereof, and modifications in which the oxides are the active agents. Particular preference is given to pigments based on titanium dioxide. It is possible to use the amounts given for the above combinations.

The cosmetic and dermatological preparations according to the invention can comprise cosmetic active substances, auxiliaries and/or additives as are customarily used in such preparations, e.g., antioxidants, preservatives, bactericides, perfumes, antifoams, dyes, pigments which have a coloring action, thickeners, surface-active substances, emulsifiers, emollients, moisturizers and/or humectants, fats, oils, waxes or other customary constituents of a cosmetic or dermatological formulation, such as alcohols, polyols, polymers, foam stabilizers, electrolytes, organic solvents or silicone derivatives.

If the cosmetic or dermatological preparation for the purposes of the present invention is a solution or emulsion or dispersion, solvents which may be used are:
water or aqueous solutions;
oils, such as triglycerides of capric or caprylic acid, but preferably castor oil;
fats, waxes and other natural and synthetic fatty substances, preferably esters of fatty acids with alcohols of low carbon number, e.g., with isopropanol, propylene glycol or glycerin, or esters of fatty alcohols with alkanoic acids of low carbon number or with fatty acids;
alcohols, diols or polyols of low carbon number, and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products.

In particular, mixtures of the abovementioned solvents are used. In the case of alcoholic solvents, water can be a further constituent.
The oil phase of the emulsions, oleogels or hydrodispersions or lipodispersions for the purposes of the present invention is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group of esters of aromatic carboxylic acids and saturated and/or unsaturated, branched and/or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

Furthermore, the oil phase can also advantageously be chosen from the group of branched and unbranched hydrocarbons and hydrocarbon waxes, silicone oils, dialkyl ethers, the group of saturated or unsaturated, branched or unbranched alcohols, and fatty acid triglycerides, namely the triglycerol esters of saturated and/or unsaturated, branched and/or unbranched alkanecarboxylic acids having a chain length of from 8 to 24 carbon atoms, in particular 12-18 carbon atoms. The fatty- acid triglycerides can, for example, be advantageously chosen from the group of synthetic, semisynthetic and natural oils, e.g., olive oil, sunflower oil, soya oil, peanut oil, rapeseed oil, almond oil, palm oil, coconut oil, palm kernel oil and the like.

Any mixtures of such oil and wax components can also be used advantageously for the purposes of the present invention. In some instances, it may also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous are mixtures of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

Advantageously, the oil phase can also have a content of cyclic or linear silicone oils, or be composed entirely of such oils, although it is preferred to use an additional content of other oil phase components apart from the silicone oil or the silicone oils.

Cyclomethicone (octamethylcyclotetrasiloxane) is advantageously used as the silicone oil to be used according to the invention. However, other silicone oils can also be used advantageously for the purposes of the present invention, for example hexamethylcyclotrisiloxane, polydimethylsiloxane, poly(methylphenylsiloxane).

Mixtures of cyclomethicone and isotridecyl isononanoate, and of cyclomethicone and 2-ethylhexyl isostearate are also particularly advantageous.

The aqueous phase of the preparations according to the invention optionally advantageously contains
alcohols, diols or polyols of low carbon number, and ethers thereof, preferably ethanol, isopropanol, propylene glycol, glycerin, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g., ethanol, isopropanol, 1,2-propanediol, glycerin and, in particular, one or more thickeners which can advantageously be chosen from the group of silicon dioxide, aluminum silicates, polysaccharides and derivatives thereof, e.g., hyaluronic acid, xanthan gum, hydroxypropylmethylcellulose, particularly advantageously from the group of polyacrylates, preferably a polyacrylate from the group of so-called Carbopols, for example, Carbopol grades 980, 981, 1382, 2984, 5984, in each case individually or in combination.

Gels used according to the invention usually contain alcohols of low carbon number, e.g., ethanol, isopropanol, 1,2-propanediol, glycerin and water or an abovementioned oil in the presence of a thickener which, in the case of oily alcoholic gels, is preferably silicon dioxide or an aluminum silicate, and, in the case of aqueous-alcoholic or alcoholic gels, is preferably a polyacrylate.

Solid sticks contain, for example, natural or synthetic waxes, fatty alcohols or fatty acid esters.

Customary bases which are suitable for use as cosmetic sticks for the purposes of the present invention are liquid oils (e.g., paraffin oils, castor oil, isopropyl myristate), semisolid constituents (e.g., Vaseline, lanolin), solid constituents (e.g., beeswax, ceresine and microcrystalline waxes or ozokerite) and high-melting waxes (e.g., carnauba wax, candelilla wax).

Suitable propellants for cosmetic and/or dermatological preparations which can be sprayed from aerosol containers for the purposes of the present invention are the customary known readily volatile, liquefied propellants, for example, hydrocarbons (propane, butane, isobutane), which can be used alone or in a mixture with one another. Compressed air can also be used advantageously.

One of skill in the art is of course aware that there are propellants which are nontoxic per se and are in principle suitable for realizing the present invention in the form of aerosol preparations, but which must nevertheless be avoided because of their unacceptable impact on the environment or other accompanying circumstances, in particular fluorinated hydrocarbons and chlorofluorohydrocarbons (CFHCs).

For the purposes of the present invention, cosmetic preparations can also be in the form of gels which, in addition to an effective content of the active substance according to the invention and solvents customarily used therefor, preferably water, also comprise organic thickeners, e.g., gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g., aluminum silicates, such as, for example, bentonites, or a mixture of polyethylene glycol and polyethylene glycol stearate or distearate. The thickener is present in the gel, for example, in an amount between 0.1 and 30% by weight, preferably between 0.5 and 15% by weight.

The following examples serve to illustrate the present invention, but not to limit it. All amounts, proportions and percentages relate to the weight and the total amount or to the total weight of the preparations, unless otherwise stated.

### EXAMPLES O/W CREAMS

### Example No. 1

| | |
|---|---|
| Glyceryl sterate self-emulsifying | 4.00 |
| Peg-40 stearate | 1.00 |
| Cetyl alcohol | 3.00 |
| Caprylic/capric triglyceride | 5.00 |
| Paraffinum liquidium | 5.00 |
| Licochalcone A | 0.05 |
| Tocopherol | 0.1 |
| Na₃HEDTA | 0.1 |
| Preservatives, perfume | q.s. |
| Polyacrylic acid | 3.00 |
| Aqueous sodium hydroxide 45% | q.s. |
| Glycerin | 5.00 |
| Water | ad 100 |

### Example No. 2

| | |
|---|---|
| Glyceryl sterate self-emulsifying | 3.00 |
| Stearic acid | 1.00 |
| Cetyl alcohol | 2.00 |
| Caprylic/capric triglyceride | 3.00 |
| Dicaprylyl ether | 4.00 |
| Paraffinum liquidium | 2.00 |
| Licochalcone A | 0.01 |
| Preservatives, perfume | q.s. |
| Polyacrylic acid | 0.1 |
| Aqueous sodium hydroxide 45% | q.s. |
| Glycerin | 3.00 |
| Butylene glycol | 3.00 |
| Water | ad 100 |

### Example No. 3

| | |
|---|---|
| Glyceryl stearate citrate | 2.00 |
| Stearyl alcohol | 2.00 |
| Lanolin alcohol | 1.00 |
| Caprylic/capric triglyceride | 4.00 |
| Paraffinum liquidium | 8.00 |
| Dimethicone | 1.00 |
| Licochalcone A | 0.04 |
| Preservatives, perfume | q.s. |
| Aqueous sodium hydroxide 45% | q.s. |
| Glycerin | 7.50 |
| Water | ad 100 |

### Example No. 4

| | |
|---|---|
| Glyceryl stearate citrate | 2.00 |
| Stearyl alcohol | 2.00 |
| Lanolin alcohol | 1.00 |
| Caprylic/capric triglyceride | 4.00 |
| Paraffinum liquidium | 8.00 |
| Dimethicone | 1.00 |
| Licochalcone A | 0.03 |
| Preservatives, perfume | q.s. |
| Aqueous sodium hydroxide 45% | q.s. |
| Glycerin | 7.50 |
| Dihydroxyacetone | 1.00 |
| Water | ad 100 |

### Example No. 5

| | |
|---|---|
| Polyglyceryl-3-methylglucose distearate | 3.00 |
| Cetyl alcohol | 3.00 |
| Caprylic/capric triglyceride | 3.00 |
| Dicaprylyl ether | 2.00 |
| Paraffinum liquidium | 3.00 |
| Licochalcone A | 0.25 |
| Na₃HEDTA | 0.1 |
| Preservatives, perfume | q.s. |
| Polyacrylic acid | 0.1 |
| Aqueous sodium hydroxide 45% | q.s. |
| Glycerin | 3.00 |
| Water | ad 100 |

### Example No. 6

| | |
|---|---|
| Glyceryl stearate citrate | 2.00 |
| Sorbitan stearate | 2.00 |
| Cetyl stearyl alcohol | 2.00 |
| Caprylic/capric triglyceride | 3.00 |
| Octyldodecanol | 2.00 |
| Dicaprylyl ether | 1.00 |
| Licochalcone A | 0.0125 |
| Tocopherol | 0.20 |
| Preservatives, perfume | q.s. |
| Polyacrylic acid | 0.1 |
| Aqueous sodium hydroxide 45% | q.s. |
| Glycerin | 3.00 |
| Water | ad 100 |

### EXAMPLES O/W CREAMS

### Example No. 7

| | |
|---|---|
| Glyceryl sterate self-emulsifying | 5.00 |
| Stearyl alcohol | 2.00 |
| Caprylic/capric triglyceride | 2.00 |
| Octyldodecanol | 2.00 |
| Dimethicone polydimethylsiloxane | 2.00 |
| Titanium dioxide | 2.00 |
| 4-Methylbenzylidene camphor | 1.00 |
| Butyl methoxydibenzoylmethane | 0.50 |
| Licochalcone A | 0.02 |
| Preservatives, perfume | q.s. |
| Polyacrylic acid | 0.15 |
| Aqueous sodium hydroxide 45% | q.s. |
| Glycerin | 3.00 |
| Water | ad 100 |

### Example No. 8

| | |
|---|---|
| Glyceryl stearate citrate | 2.00 |
| Cetyl stearyl alcohol | 3.00 |
| C12-15 alkyl benzoate | 2.00 |
| Octyldodecanol | 2.00 |
| Paraffinum liquidum | 4.00 |
| Licochalcone A | 0.125 |
| 2,4-Bis-(4-(2-ethylhexyloxy)-2-hydroxyl)-phenyl)-6-(4- | 1.0 |
| methoxyphenyl)-(1,3,5)-triazine | |
| Dihydroxyacetone | 0.5 |
| Preservatives, perfume | q.s. |
| Polyacrylic acid | 0.1 |
| Aqueous sodium hydroxide 45% | q.s. |
| Butylene glycol | 3.00 |
| Ethanol | 3.00 |
| Water | ad 100 |

### Example No. 9

| | |
|---|---|
| Glyceryl stearate citrate | 2.00 |
| Cetyl stearyl alcohol | 1.00 |
| C12-15 alkyl benzoate | 3.00 |
| Paraffinum liquidum | 2.00 |
| Licochalcone A | 0.05 |
| 2,4-Bis-(4-(2-ethylhexyloxy-)2-hydroxyl)-phenyl)-6-(4- | 3.0 |
| methoxyphenyl)-(1,3,5)-triazine | |
| Ethylenediaminetetraacetic acid trisodium | 0.20 |
| Preservatives, perfume | q.s. |
| Xanthan gum | 0.20 |
| Aqueous sodium hydroxide 45% | q.s. |
| Glycerin | 3.00 |
| Water | ad 100 |

### Example No. 10

| | |
|---|---|
| Stearic acid | 2.50 |
| Cetyl alcohol | 3.00 |
| Octyldodecanol | 4.00 |
| Cyclic dimethylpolysiloxane | 0.50 |
| Licochalcone A | 0.2 |
| Preservatives, perfume | q.s. |
| Polyacrylic acid | 0.05 |
| Aqueous sodium hydroxide 45% | q.s. |
| Glycerin | 5.00 |
| Ethanol | 3.00 |
| Water | ad 100 |

### Example No. 11

| | |
|---|---|
| Stearic acid | 3.50 |
| Cetyl alcohol | 4.50 |
| Cetylstearyl alcohol | 0.50 |
| Octyldodecanol | 6.00 |
| Cyclic dimethylpolysiloxane | 2.00 |
| 4-Methylbenzylidene camphor | 1.00 |
| Butylmethoxy-dibenzoylmethane | 0.50 |
| Licochalcone A | 0.10 |
| 2,4-bis-(4-(2-ethylhexyloxy-)2-hydroxyl)-phenyl)-6-(4- | 0.5 |
| methoxyphenyl)-(1,3,5)-triazine | |
| Dihydroxyacetone | 0.5 |
| Tocopherol | 0.05 |
| Ethylenediaminetetraacetic acid trisodium | 0.20 |
| Preservatives, perfume | q.s. |
| Polyacrylic acid | 0.05 |
| Aqueous sodium hydroxide 45% | q.s. |
| Glycerin | 3.00 |

### EXAMPLES W/O EMULSIONS

### Example No.12

| | |
|---|---|
| Polyglyceryl-2-dipolyhydroxystearate | 5.00 |
| 2,4-Bis-(4-(2-ethylhexyloxy-)2-hydroxyl)-phenyl)-6-(4- | 2.00 |
| methoxyphenyl)-(1,3,5)-triazine | |
| Diethylhexyl butamidotriazone | 3.00 |
| Octocrylene | 7.00 |
| Diethylhexyl butamidotriazone | 1.00 |
| Phenylene-1,4-bis-(monosodium,-2-benzimidazyl-5,7- | 1.00 |
| disulfonic acid) | |
| Phenylbenzimidazole sulfonic acid | 0.50 |
| Zinc oxide | 3.00 |
| Dicaprylylether | 10.00 |
| Dicaprylyl carbonate | 5.00 |
| Phenylmethylpolysiloxane | 2.00 |
| PVP hexadecene copolymer | 0.50 |
| Glycerin | 3.00 |
| Magnesium sulfate | 1.00 |
| Tocopherol acetate | 0.50 |
| Licochalcone A | 0.05 |
| Preservatives, perfume | q.s. |
| Ethanol | 3.00 |
| Water | ad 100 |

### Example No. 13

| | |
|---|---|
| Cetyldimethicone copolyol | 2.50 |
| 2-Ethylhexyl methoxycinnamate | 8.00 |
| 2,4-Bis-(4-(2-ethylhexyloxy-)2-hydroxyl)-phenyl)-6-(4- | 2.50 |
| methoxyphenyl)-(1,3,5)-triazine | |
| Diethylhexyl butamidotriazone | 1.00 |
| 4-Methylbenzylidene camphor | 2.00 |
| Octocrylene | 2.50 |
| Phenylene-1,4-bis-(monosodium,-2-benzimidazyl-5,7- | 2.00 |
| disulfonic acid) | |
| Titanium dioxide | 2.00 |
| Zinc oxide | 1.00 |
| Dimethicone polydimethylsiloxane | 4.00 |
| Phenylmethylpolysiloxane | 25.00 |
| Octoxyglycerin | 0.30 |
| Glycerin | 7.50 |
| Glycin soy | 1.00 |
| Magnesium sulfate | 0.50 |
| Licochalcone A | 0.02 |
| Preservatives, perfume | q.s. |
| Water | ad 100 |

### Example No. 14

| | |
|---|---|
| PEG-30-dipolyhydroxystearate | 5.00 |
| Butylmethoxy-dibenzoylmethane | 2.00 |
| Ethylhexyl triazone | 3.00 |
| Octocrylene | 4.00 |
| Phenylene-1,4-bis(monosodium,-2-benzimidazyl-5,7-disulfonic | 0.50 |
| acid | |
| Titanium dioxide | 1.50 |
| Zinc oxide | 2.00 |
| Paraffinum liquidum | 10.0 |
| Butylene-glycol-dicaprylate/-dicaprate | 2.00 |
| Dicaprylyl carbonate | 6.00 |
| Dimethicone polydimethylsiloxane | 1.00 |
| Shea butter | 3.00 |
| Octoxyglycerin | 1.00 |
| Glycin soy | 1.50 |
| Magnesium chloride | 1.00 |
| Tocopherol acetate | 0.25 |
| Licochalcone A | 0.125 |
| Preservatives, perfume | q.s. |
| Ethanol | 1.50 |
| Water | ad 100 |

### Example No. 15

| | |
|---|---|
| Cetyldimethicone copolyol | 4.00 |
| 2-Ethylhexyl methoxycinnamate | 5.00 |
| 2,4-Bis-(4-(2-ethylhexyloxy-)2-hydroxyl)-phenyl)-6-(4- | 2.00 |
| methoxyphenyl)-(1,3,5)-triazine | |
| Butylmethoxy-dibenzoylmethane | 1.00 |
| Ethylhexyl triazone | 4.00 |
| 4-Methylbenzylidene camphor | 4.00 |
| Diethylhexyl butamidotriazone | 2.00 |
| Phenylbenzimidazole sulfonic acid | 3.00 |
| Zinc oxide | 0.50 |
| C₁₂₋₁₅ Alkyl-benzoate | 9.00 |
| Butylene-glycol-dicaprylate/-dicaprate | 8.00 |
| Dimethicone polydimethylsiloxane | 5.00 |
| PVP hexadecene copolymer | 0.50 |
| Glycerin | 7.50 |
| Magnesium sulfate | 0.50 |
| Licochalcone A | 0.20 |
| Preservatives, perfume | q.s. |
| Water | ad 100 |

### Example No. 16

| | |
|---|---|
| Polyglyceryl-2-dipolyhydroxystearate | 4.50 |
| 2-Ethylhexyl methoxycinnamate | 4.00 |
| 2,4-Bis-(4-(2-ethylhexyloxy-)2-hydroxyl)-phenyl)-6-(4- | 2.50 |
| methoxyphenyl)-(1,3,5)-triazine | |
| Diethylhexyl butamidotriazone | 3.00 |
| Ethylhexyl triazone | |
| 4-Methylbenzylidene camphor | 2.00 |
| Octocrylene | 2.50 |
| Phenylbenzimidazol sulfonic acid | 2.00 |
| Titanium dioxide | 3.00 |
| Paraffinum liquidum | 8.00 |
| Dicaprylylether | 7.00 |
| Butylene-glycol-dicaprylate/-dicaprate | 4.00 |
| Phenylmethylpolysiloxane | 2.00 |
| PVP hexadecene copolymer | 1.00 |
| Octoxyglycerin | 0.50 |
| Glycerin | 2.50 |
| Magnesium chloride | 0.70 |
| Tocopherolacetate | 1.00 |
| Licochalcone A | 0.25 |
| Preservatives, perfume | q.s. |
| Ethanol | 1.00 |
| Water | ad 100 |

### EXAMPLES W/O EMULSIONS

### Example No. 17 18

| | | |
|---|---|---|
| Polyglyceryl-2-dipolyhydroxystearate | 4.00 | 5.00 |
| Lanolin alcohol | 0.50 | 1.50 |
| Isohexadecane | 1.00 | 2.00 |
| Myristyl-myristate | 0.50 | 1.50 |
| Vaseline | 1.00 | 2.00 |
| Butylmethoxy-dibenzoylmethane | 0.50 | 1.50 |
| 4-Methylbenzylidene camphor | 1.00 | 3.00 |
| Butylene-glycol-dicaprylate/-dicaprate | 4.00 | 5.00 |
| Shea butter | — | 0.50 |
| Butylene glycol | — | 6.00 |
| Octoxyglycerin | — | 3.00 |
| Glycerin | 5.00 | — |
| Tocopherol acetate | 0.50 | 1.00 |
| Licochalcone A | 0.2 | 0.1 |
| EDTA | 0.20 | 0.20 |
| Preservatives | q.s. | q.s. |
| Ethanol | — | 3.00 |
| Perfume | q.s. | q.s. |
| Water | ad 100 | ad 100 |

### EXAMPLE (W/O CREAM)

### Example No. 19

| | |
|---|---|
| Polyglyceryl-3-diisostearate | 3.50 |
| Glycerin | 3.00 |
| Polyglyceryl-2-dipolyhydroxystearate | 3.50 |
| Licochalcone A | 0.1 |
| Preservatives | q.s. |
| Perfume | q.s. |
| Magnesium sulfate | 0.6 |
| Isopropylstearate | 2.0 |
| Caprylylether | 8.0 |
| Cetearyl isononanoate | 6.0 |
| Water | ad 100 |

### EXAMPLE (W/O EMULSION)

### Example No. 20

| | |
|---|---|
| Triceteareth-4-phosphate | 0.80 |
| Butylated hydroxytoluene | 0.05 |
| Glyceryl lanolate | 1.70 |
| Cyclomethicone | 2.20 |
| Isopropyl palmitate | 1.00 |
| Licochalcone A | 0.10 |
| Polyacrylic acid | 0.50 |
| Ethylenediaminetetraacetic acid | 1.00 |
| Sodium hydroxide | q.s. |
| Citric acid | 0.01 |
| Preservatives | q.s. |
| Perfume | q.s. |
| Water | ad 100 |

1.-3. (canceled)

4. A method for treating postinflammatory skin conditions, wherein the method comprises applying to skin exhibiting a postinflammatory skin condition a cosmetic or dermatological preparation that comprises licochalcone A.

5. The method of claim 4, wherein the preparation comprises an extract of radix glycyrrhizae inflatae that comprises licochalcone A.

6. The method of claim 4, wherein the preparation comprises from 0.0001% to 5% by weight of licochalcone A, based on a total weight of the preparation.

7. The method of claim 6, wherein the preparation comprises from 0.001% to 1% by weight of licochalcone A.

8. The method of claim 6, wherein the preparation comprises from 0.005% to 0.15% by weight of licochalcone A.

9. The method of claim 4, wherein the preparation further comprises one or more polyols.

10. The method of claim 6, wherein the preparation further comprises from 0.001% to 10% by weight of one or more polyols, based on a total weight of the preparation.

11. The method of claim 10, wherein the preparation comprises from 0.05% to 5% by weight of one or more polyols.

12. The method of claim 1 1, wherein the preparation comprises from 0.01% to 2% by weight of one or more polyols.

13. The method of claim 12, wherein the one or more polyols comprise butylene glycol.

14. The method of claim 4, wherein the postinflammatory skin condition comprises hyperpigmentation.

15. The method of claim 4, wherein the postinflammatory skin condition comprises hypopigmentation.

16. A method for the prophylaxis of postinflammatory skin conditions, wherein the method comprises applying to postinflammatory skin a cosmetic or dermatological preparation that comprises licochalcone A.

17. The method of claim 16, wherein the preparation comprises an extract of radix glycyrrhizae inflatae that comprises licochalcone A.

18. The method of claim 16, wherein the preparation comprises from 0.0001% to 5% by weight of licochalcone A, based on a total weight of the preparation.

19. The method of claim 18, wherein the preparation comprises from 0.001% to 1% by weight of licochalcone A.

20. The method of claim 18, wherein the preparation comprises from 0.005% to 0.15% by weight of licochalcone A.

21. The method of claim 16, wherein the preparation further comprises one or more polyols.

22. The method of claim 18, wherein the preparation further comprises from 0.001% to 10% by weight of one or more polyols, based on a total weight of the preparation.

23. The method of claim 22, wherein the preparation comprises from 0.05% to 5% by weight of one or more polyols.

24. The method of claim 23, wherein the preparation comprises from 0.01% to 2% by weight of one or more polyols.

25. The method of claim 24, wherein the one or more polyols comprise butylene glycol.

26. A cosmetic or dermatological preparation for treatment or prophylaxis of postinflammatory skin conditions, wherein the preparation comprises licochalcone A and is associated with instructions directing use of the preparation for at least one of treatment and prophylaxis of a postinflammatory skin condition.
